(19) United States
(12) Patent Application Publication
(10) Pub. No.: US 2007/0292366 A1
(43) Pub. Date: Dec. 20, 2007

(54) SYSTEM, KIT AND METHOD FOR PROMOTING AND MAINTAINING ORAL HEALTH

(76) Inventors: Tim Clarot, Phoenix, AZ (US); Regina Miskewitz, Phoenix, AZ (US)

Correspondence Address: SNELL & WILMER L.L.P. (Main), 400 EAST VAN BUREN, ONE ARIZONA CENTER, PHOENIX, AZ, 85004-2202, US

(21) Appl. No.: 11/749,106

(22) Filed: May 15, 2007

Related U.S. Application Data
(60) Provisional application No. 60/800,638, filed on May 15, 2006.
(60) Provisional application No. 60/800,631, filed on May 15, 2006.

(51) Int. Cl. A61K 8/21; A61K 8/18; A61K 8/22; A61K 8/23; A61K 8/24; A61K 8/27; A61K 8/97; A61Q 11/00

(52) U.S. Cl. 424/52; 424/49; 424/53; 424/56; 424/57; 424/58

(57) ABSTRACT

A composition for improving oral health, a system including the composition, a method of using the composition and system, and a method of forming the composition and system are disclosed. The composition is a gel that maintains contact with a portion of an oral cavity for an extended period of time to, for example, retard plaque and calculus buildup. The system includes a composition and a container encapsulating the composition. The composition is applied by rupturing the container and applying the composition through the ruptured portion of the container.

### CROSS REFERENCE TO RELATED APPLICATIONS

This application claims the benefit of U.S. patent application Ser. No. 60/800,638, entitled METHOD AND SYSTEM FOR FACILITATING ORAL HEALTH, filed May 15, 2006, and U.S. patent application Ser. No. 60/800,631, entitled METHOD AND SYSTEM FOR FACILITATING AND MAINTAINING ORAL HEALTH THROUGH PRESCRIBED APPLICATIONS OF ORAL COMPOSITIONS, filed May 15, 2006.

### FIELD OF INVENTION

The present invention generally relates to systems and methods for promoting and maintaining oral health and hygiene. More particularly, the invention relates to kits and systems including a composition for facilitating oral health, such as through the reduction and/or prevention of tartar, plaque, gingivitis, and other oral diseases and to methods of using the systems and kits.

### BACKGROUND OF THE INVENTION

Unfortunately, poor oral health affects millions of people every year. Poor oral health may result in symptoms ranging from bad breath, tooth decay, and tooth coloration, to more serious health problems, such as gum disease, tooth loss, and even general health problems, such as heart disease, stroke, poorly controlled diabetes and preterm labor.

The presence of dental plaque, or simply plaque, in an oral cavity can lead to such oral and general health problems. Plaque can be defined as an organized, coherent, gel-like or mucoid material that includes microorganisms in an organic matrix derived from saliva and extracellular bacterial products such as glucans, fructans, enzymes, toxins, and acids. Plaque may also contain other cells, such as desquamated epithelial cells, and inorganic components, such as calcium and phosphate. In general, dental plaque is a bacterial accumulation. Generally transparent and sticky, plaque accumulates around the teeth at the cervical margin, and then grows apically.

Once plaque forms on a surface, the plaque resists removal, and usually can be removed only by mechanical means, such as, for example, by brushing and flossing the affected areas. If not removed, however, the presence of plaque can give rise to tartar formation, tooth decay, gingivitis, periodontitis, and other health problems.

Tartar is a hard, calcified plaque material that exhibits a yellowish or brownish color. Tartar forms as a result of minerals (e.g., those present in saliva and gum pockets) reacting with plaque material to form a rough calculus. Calculus generally arises from the nucleation of calcium phosphate, often in areas where the large salivary gland ducts secrete their saliva. As such, calculus can form on surfaces not covered by the oral mucosa (supragingival) or on surface located apical to the soft tissue margin of the gingiva (subgingival).

Tartar adheres to hard surfaces such as enamel, roots, and dental devices, such as dentures, bridges, crowns, and the like, and is generally more difficult to remove than plaque. Brushing and flossing are normally not sufficient to remove tartar from a surface.

If left untreated, tartar buildup can be problematic in several regards. For example, the rough, porous surface of tartar serves as a breeding ground for additional bacteria, which can calcify and form additional tartar. The bacteria growth can, in turn, lead to tooth decay, gum disease, tooth loss, as well as systemic health problems.

In addition to the health concerns, tartar is a cosmetic problem due to its discoloration of teeth. Namely, teeth can become yellowish or brownish color. Moreover, because the surface of tartar is rough and porous, the tartar absorbs colors from other sources (e.g., coffee, tea, tobacco, smoke, red wine and the like), and thus the presence of tartar exacerbates cosmetic tooth coloration typically associated with such other sources.

Typical methods of preventing tartar buildup include brushing with a tartar control toothpaste. Although such toothpastes, if used regularly, may prevent additional buildup of tartar, the toothpastes are not thought to be effective at removing existing tartar from tooth and device surfaces.

Methods of removing existing tartar typically include scaling or root planing, both of which are performed by dentists or hygienists with the aid of specialized tools. Although these techniques work well, they are relatively expensive and time consuming. Furthermore, various methods for inhibiting tartar may cause damage to tooth enamel and/or to dental devices.

Accordingly, improved systems and techniques for removing existing tartar and plaque and for reducing an amount of plaque and tartar buildup are desired.

### SUMMARY OF THE INVENTION

In accordance with various aspects of the present invention, a kit, system and method for promoting and maintaining oral health are provided. The various exemplary oral health care kits, systems and methods provide a relatively inexpensive and safe treatment for facilitating maintenance and improvement of oral health and/or hygiene, such as through the prevention and/or reduction of tartar, plaque, gingivitis, and other diseases. In addition, the various exemplary oral health care kits, systems and methods are relatively easy to use or perform, do not require a visit to a dentist office, and do not damage the surface of enamel.

In accordance with various exemplary embodiments of the invention, a system for facilitating oral health comprises a composition configured to prevent and/or reduce tartar, plaque, gingivitis, gum bleeding, periodontitis, and/or otherwise facilitate oral health or hygiene, and a container configured for expulsion and application of the composition to a portion of an oral cavity, such as gums, teeth, gingival surface, interfaces thereof, and/or any dental device. In accordance with additional aspects of these embodiments, the container encapsulates multiple doses of the composition. Alternatively, a system includes a container for encasing a single dose. In accordance with yet further aspects, the container is configured to facilitate application of the composition to specific areas within an oral cavity.

In accordance with various embodiments of the invention, a container is configured for application of the composition to an oral cavity. In accordance with particular examples, the container includes an expulsion or vessel portion configured to store and facilitate expulsion or other like transfer of the composition and an applicator portion configured to receive the composition and to facilitate delivery of the composition to a surface of the oral cavity. In accordance with various exemplary embodiments, the applicator portion includes an angled spout to facilitate targeted delivery of the composition—e.g., to a gum/tooth interface. In accordance with further aspects, a container is configured with an access/closure portion to maintain the composition within the container. In accordance with additional aspects of this embodiment, the container encapsulates multiple doses of the composition. Alternatively, a system includes a container for encasing a single dose.

In accordance with one embodiment of the invention, a system includes a viscous composition, including at least one active ingredient to promote and/or maintain oral health and a container configured to dispense the viscous composition. In accordance with various aspects of this embodiment, the viscous composition is designed to maintain the active ingredient(s) in contact with a surface for an extended period of time. Exemplary compositions have a viscosity greater than about 20,000 centipoise (cp), preferably greater than about 30,000 cp, and more preferably greater than about 35,000 cp. The viscosity of the compositions may range from about 20,000 to about 250,000 cp, preferably about 25,000 to about 100,000, and more preferably about 30,000 to about 50,000 cp. Suitable active ingredients include cetylpyridinium chloride (CPC), zinc salts, other antimicrobial agents, and other ingredients to improve oral health.

In accordance with another embodiment of the invention, a system includes a composition, having a plurality of active ingredients to improve oral health, and a container to hold the composition. In accordance with various aspects of this embodiment of the invention, the composition includes a carrier having a thickening agent, wherein the composition is configured to maintain the plurality of active ingredients in contact with a surface for an extended period of time. Exemplary thickening agents suitable for use in the composition include hydroxyethylcellulose and other pharmaceutically acceptable thickeners, and exemplary active ingredients include CPC, zinc salts, other antimicrobial agents, and similar agents known to reduce or prevent buildup of tartar, plaque gingivitis, gum bleeding, periodontitis, or other disease and/or the effects of the same.

In accordance with a further embodiment of the invention, a system includes a composition, having one or more active ingredients to improve oral health and a carrier including a thickening agent, and a container to hold the composition. Exemplary thickening agents suitable for use in the composition include hydroxyethylcellulose, glycerin, and other pharmaceutically acceptable thickeners, and exemplary active ingredients include CPC, zinc salts, other antimicrobial agents, and similar agents known to improve oral health.

In accordance with additional embodiments of the invention, a kit includes one or more systems.

In accordance with further exemplary embodiments of the invention, a method of facilitating oral health care includes providing a system, including a composition within a container, accessing the composition, and selectively applying the composition to a surface within an oral cavity.

In accordance with further exemplary embodiments of the invention, a method of facilitating oral health care includes providing a kit, including a system having a composition within a container, accessing the composition, and selectively applying the composition to a surface within an oral cavity.

### BRIEF DESCRIPTION OF THE DRAWINGS

The exemplary embodiments of the present invention will be described in connection with the appended drawing figures in which like numerals denote like elements and:

FIG. 1 illustrates an exemplary system for facilitating oral health care in accordance with an exemplary embodiment of the invention;

FIG. 2 illustrates another exemplary system for expulsion and application of a composition to an oral cavity in accordance with an exemplary embodiment of the invention;

FIG. 3 illustrates yet another system in accordance with various embodiments of the invention;

FIGS. 4A-4C illustrate, respectively, a left view, a front view, and a right view of an exemplary container system in accordance with an exemplary embodiment of the invention;

FIG. 5 illustrates a kit including a plurality of systems, in accordance with yet another embodiment of the invention; and

FIG. 6 illustrates a block diagram of an exemplary method for facilitating oral health care in accordance with an exemplary embodiment of the invention.

Elements in the figures are illustrated for simplicity and clarity and have not necessarily been drawn to scale. The dimensions of some of the elements in the figures may be exaggerated relative to other elements to help to improve understanding of embodiments of the present invention.

### DETAILED DESCRIPTION

The present invention provides an oral care system and kit to improve oral health and methods of using and forming the system and kit. The system, kit, and method of the invention can be used to improve oral health of various animals, and are particularly well suited for the treatment of humans.

The invention is described herein in terms of various functional components, compositions and processing steps. It should be appreciated that such components, compositions and steps may be realized by any number of structural components and compositional constituents configured to perform the specified functions. For example, the present invention may employ various compositions and containers for use with systems and kits for promoting oral health care; the specific examples as described herein are merely indicative of exemplary applications for the invention.

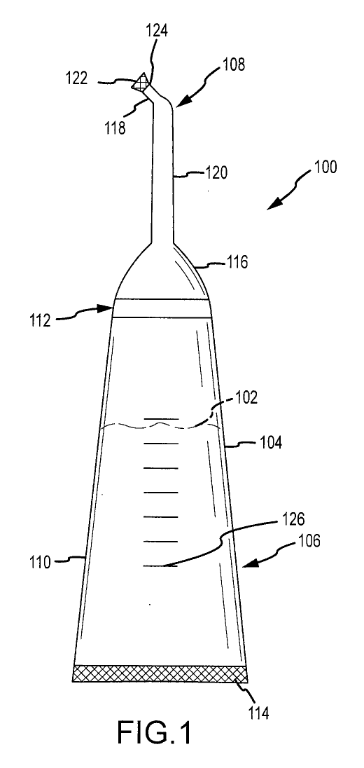

FIG. 1 illustrates a system 100 in accordance with various embodiments of the present invention. System 100 is used to facilitate prevention and/or reduction of tartar, plaque, gingivitis, other diseases, and/or otherwise improve oral health and hygiene.

In accordance with various exemplary aspects of the illustrated embodiment, system 100 includes a composition 102 and a container 104 for applying composition 102 to a surface within an oral cavity.

As used throughout this application, the term “surface” includes any surface on which plaque, tartar, or gum disease may form. Exemplary surfaces include teeth (both supragingival and subgingival), gums, and dental devices such as bridges, crowns, fillings, braces, and the like. Further, as used herein, the term “measurably improve” means a measurable difference between an amount measured without use of the composition or system of the present invention and with or after use of the system. The measurements may be compared for the same surface (before and after) or between test and control groups.

Container 104 is configured for containment and temporary storage of composition 102, i.e., storage until initiation of the treatment process, and for expulsion and application of composition 102 to a surface to achieve improved oral health and/or hygiene. Container 104 can be configured in various manners for application of composition 102 to a surface. For example, container 104 can comprise various sizes and volumes depending on treatment applications, and/or various shapes and configurations for facilitating delivery of composition 102 to a surface, depending upon, for example, the purpose for which composition 102 is being applied. In the case of prophylaxis or reduction in plaque, calculus, gingivitis, and the like, it may be desirable to have a multi-dose applicator for convenient, repeated (e.g., daily) application of composition 102. In contrast, a single-dose applicator may be desirable for travel or for applications to specific problem areas, such as targeted application to diseased or infected areas or dental devices within an oral cavity.

In accordance with specific examples of various embodiments, container 104 is configured to store about seven doses, about four doses, about two doses, or about one dose. However, the invention is not necessarily limited to these container sizes.

A dose size may vary in accordance with several factors, such as the particular ingredients, the dilution of the composition, and the like. Exemplary dose sizes for purpose of illustration range from about 1 mg to about 6 mg, preferably about 2 mg to about 5 mg, and more preferably about 3 mg to about 4 mg.

With continued reference to FIG. 1, container 104 includes an expulsion or vessel portion 106 configured to contain or store composition 102 and to facilitate expulsion of composition 102, and an applicator portion 108 configured to receive composition 102 from expulsion portion 106 and to facilitate application or delivery of composition 102 to a selected oral cavity surface.

In accordance with particular aspects of this embodiment, portion 106 is formed of a resiliently deformable material that is capable of retaining and returning to its original shape when not under pressure. In accordance with other aspects, portion 106 is formed of material that does return to its original shape. Exemplary resilient materials suitable for portion 106 include low density polyethylene material, high density polyethylene, medium density polyethylene, linear low density polyethylene, polyvinyl chloride, K resin, polyethylene terephthalate and copolyesters, polypropylene, surlyn, silicones and other thermostatics, metal or alloy, and the like. Portion 106 may be opaque, transparent, or semitransparent. An advantage of forming vessel portion 106 of transparent or semitransparent material is that an amount of material 102 within vessel portion 106 can be ascertained when the portion is formed of such material. Material used to form vessel portion 106 may also include UV protection additives, colorants, or the like, and is preferably FDA-approved material.

In accordance with various embodiments of the invention, expulsion or vessel portion 106 includes a resilient vial 110 and a neck 112. Resilient vial 110 acts as a reservoir for material 102 and also facilitates expulsion of material 102 from system 100 when pressure is applied to an external surface of vial 110. As illustrated, vial 110 may also include graduations 126 to, for example, illustrate a number of doses used and/or a number of doses remaining. Neck 112 is configured to couple to applicator or spout portion 108.

Although illustrated as substantially tubular, with a sealed end 114, resilient vial 110 may be of any suitable shape. For example, vial 110 may be pyramidal, cone shaped, fluted, or have a rectangular cross section. Similarly, end portion 114 may be of any suitable shape, such as linear (e.g., a crimped or heat-sealed end) or the like. In general, preferred shapes of vial 110 conserve material used to form the vial, allow for easy dispensing of material 102, are easy to produce, and produce relatively little scrap during production.

Container 104 can also be configured to allow a user to suitably control the rate of expulsion into applicator portion 108. For example, in accordance with an exemplary embodiment, container 104 includes a transition region 116 to facilitate flow between vial 110 and spout 108. Transition portion 116 may be of any suitable shape such as frustro-conical, fluted, semi-spherical, and the like.

Applicator portion 108 may be formed of any of the materials described above in connection with portion 106. Portion 108 is suitably configured for selective or otherwise controlled delivery of composition 102 to a target area, for example within an oral cavity. In accordance with various embodiments of the invention, applicator portion 108 is configured to couple (e.g., detachably or otherwise) to expulsion portion 106. Alternatively, portion 108 is configured as a molded or otherwise unitary structure with expulsion portion 106, as described in more detail below. When separately formed, portions 106 and 108 may be coupled using screwed, press-fit, clamped or other techniques to permanently, semi-permanently or removably attached portions 106 and 108.

In accordance with an exemplary embodiment, applicator portion 108 comprises a structure 120 to allow composition 102 to be forced through an applicator tip 118 and onto a surface within an oral cavity. Structure 120 may be passive and substantially rigid to allow composition to flow from portion 106 to tip 118. Alternatively, structure 120 and/or applicator portion 108 may be configured as less-rigid to allow for expulsion of any remaining composition within applicator portion 108 to be squeezed or otherwise delivered or applied by applicator tip 118 onto a surface.

As illustrated structure 120 may form an angle of about zero degrees with respect to a centerline through expulsion portion 106. Alternatively, structure 120 may form other angles, ranging from about zero degrees to about 90 degrees. In further accordance with the illustrated embodiment, tip 118 forms an angle of about 45 degrees relative to the centerline of component 120; however, tip 118 may suitably form other angles relative to component 120.

Tip 118 is generally configured to facilitate placement and/or controlled flow of material dispensed from system 100. Tip 118 may be substantially cylindrical. Alternatively, tip 118 may have a square, rectangular, ellipse, or other cross-sectional configuration. Tip 118 may also include a weakened section 124, which may be formed, for example, by scarring a portion of tip 118. Weakened section 124 may facilitate rupturing tip 118 at weakened section 124, which in turn allows for a predicable cross section of tip 118 and thus a relatively predictable flow of material dispensed from system 100. In accordance with one particular example, tip 118 includes a substantially constant cross-section, which makes the flow more predictable, even if tip 118 is ruptured away from weakened area 124.

End portion 122 forms a sealed end at one end of tip 118. In accordance with various embodiments of the invention, end portion 122 is flat and wider at an exterior portion than an interior portion, such that end portion 122 is wider in at least one direction than tip 118. Having end portion 122 wider than tip 118 allows a user to grip end 122 to, for example, tear or sever tip 118 at weakened section 124. However, end 122 may be alternatively configured as, for example, a semisphere or other suitable shape. Alternatively, tip 118 and end portion 122 may be configured, such that end 122 can reattach to tip 118.

Spout portion 108 may be formed using a variety of materials, such as any of the materials described above in connection with vessel portion 106. However, because spout portion 108 may be formed separately from vessel portion 106, it need not be formed of the same material.

In accordance with one embodiment of the invention, vessel portion 106 and spout portion 108 are configured to sealably (and optionally rotatably) couple to each other. In the illustrated embodiment, portion 106 and 108 are threadedly coupled to each other. In accordance with other embodiments, one of portions 106 and 108 includes a protrusion and the other of portion 106, 108 includes a recess to receive the protrusion, such that the protrusion and recess hold vessel portion 106 and spout portion 108 together, while optionally allowing the two portions to rotate about an axis, with respect to each other. In accordance with another embodiment of the invention, portions 106, 108 are configured to allow vessel portion 106 and spout portion 108 to be detachably coupled to each other. In this case, neck 112 and spout 108 may be snap-fit together as described above, or portions 106 and cap 108 may engage using lug or interference-fit technology to sealably attach to each other.

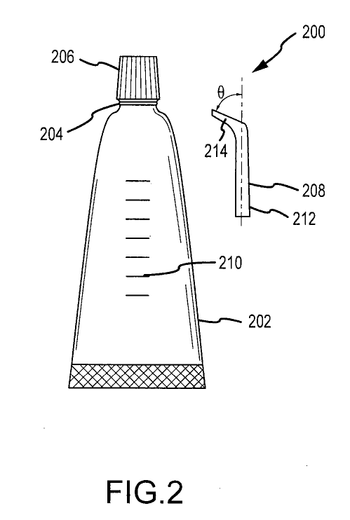

FIG. 2 illustrates another system 200 for use in accordance with additional embodiments of the invention. As illustrated, system 200 includes a vessel portion 202, including a neck 204, a cap 206, and a detachable applicator 208. System 200 is similar to system 100, except system 200 includes resealable cap 206 and detachable applicator 208, rather than applicator portion 108. System 200 may be formed of any of the materials noted above in connection with system 100, and may include graduations 210 to indicated a number of doses used and/or a number of remaining doses, as described above.

Cap 206 can be removably attached to vessel portion 202 using a variety of techniques. For example, cap 206 may be threadedly attached to portion 202. Alternatively, cap 206 and portion 202 may be coupled using snap-fit, lug, interference-fit technology, or similar technologies. In accordance with one specific example of this embodiment, neck 204 includes exterior threads and cap 206 includes interior threads to threadedly engage with neck 204.

Similarly, applicator portion 208 may couple to vessel 202 in a variety of ways, such as threaded, snap-fit, lug, or similar type connections. By way of particular example, applicator 208 threadedly engages with an interior portion of neck 204.

Applicator 208 includes a first portion 212 and a second or tip portion 214. As illustrated, tip portion 214 is angled relative to a centerline first portion 212; however such is not required for practice of the present invention. Exemplary angles range from about zero to about ninety degrees, and one particular exemplary angle is about forty-five degrees relative to the centerline.

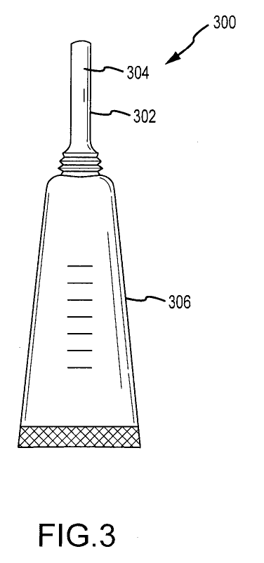

FIG. 3 illustrates yet another system 300 in accordance with additional embodiments of the invention. System 300 is similar to system 200, except system 300 includes an applicator 302, rather than applicator 208. Applicator 302 is similar to applicator 208, except applicator 302 does not include an angled tip.

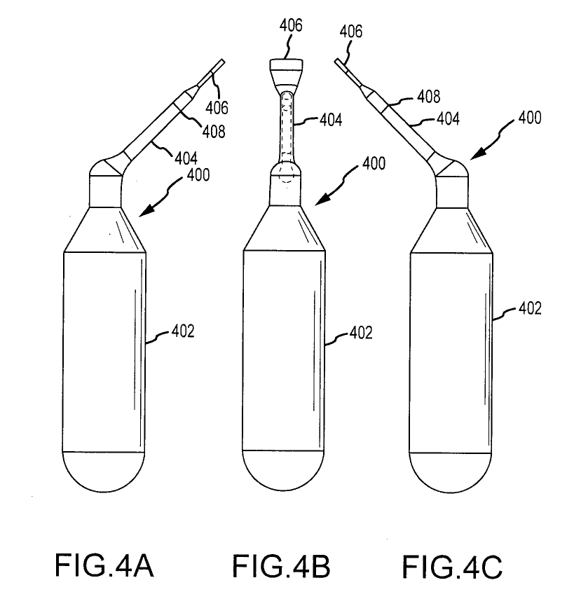

FIGS. 4A-4C illustrate left, front, and right views of yet another system 400 in accordance with various embodiments of the invention. System 400 is similar to systems 100-300, except system 400 is designed as a unitary system, having an integrated vessel 402 and spout 404, including a severable end 406 and scarred section 408.

Although not illustrated, systems in accordance with various embodiments of the invention may include tamper-resistant features. For example, system 100 may include a seal formed over neck 112, using, for example plastic or foil glued to or otherwise adhered to a top portion of neck 112. Alternatively, after spout portion 108 is attached to vessel portion 106, the two portions may be fused together using heat sealing and/or adhesive techniques.

In accordance with various exemplary embodiments, a composition (e.g., composition 102), suitable for use with systems (e.g., systems 100, 200, 300) of the present invention comprises one or more active ingredients and a viscous carrier. In this case, composition 102 is configured to maintain the active ingredient(s) in contact with a surface of an oral cavity for an extended period of time to allow the active ingredient(s) to remain in contact with the surface for an extended period.

Exemplary active ingredients suitable for use with systems of the invention include one or more of the following: cetylpyridinium chloride (CPC), dicalcium phosphate dehydrate, hydrogen peroxide, sanguinaria extract, sodium bicarbonate, sodium lauryl sulfate, sodium fluoride, stannous fluoride, sodium monofluorophosphate (MFP), zinc salts such as zinc chloride, zinc acetate, zinc citrate, zinc oxide and zinc gluconate, alkyl dimethyl amine oxide, alkyl dimethyl glycine, eucalyptol, menthol, methyl salicylate, thymol, sodium citrate, peppermint oil, sage oil, polymethylsiloxane, polxamer, and stannous pyrophosphate. Other now known or hereafter devised actives may also be used. For example, any agent, which alone or in combination is able to prevent or alleviate the severity of problems associated with dentition may be utilized. Such may include anti-caries agents and the like; agents useful in reducing tooth hypersensitivity, such as potassium nitrate, strontium chloride and/or the like; and/or plaque and calculus reducing agents, such as, for example, chlorhexidine, quaternary ammonium compounds (e.g. benzethonium chloride, domphen bromide, etc.), triclosan, herbal compounds (e.g. sanguinarine), stannous salts, complex phosphates (e.g., pyrophosphates), SLS (e.g. sodium lauryl sulfate), hydrogen peroxide, and/or the like.

An amount of the active ingredient for use within compositions suitable for uses with the invention varies in accordance with the dosage size and particular ingredient(s). In general, each active or actives selected will be used in a suitably effective amount, generally on the order of less than about 10 wt %, and more preferably 5 wt % or less. An amount of active may also be desirably selected to be within government guidelines, such as guidelines by the Food and Drug Administration in the USA. In particularly preferred compositions, the active ingredient is present in an amount of about 0.001 wt % to about 1.5 wt %, within an amount of about 0.025 wt % to about 1.0 wt %, or even within an amount about 0.05 wt % to about 0.7 wt %. All percentages set forth herein are in weight percent of the total composition, unless otherwise indicated.

In accordance with one preferred exemplary embodiment, the active ingredient(s) include CPC. In one case, CPC is present in an amount of about 0.001% to about 1%, in an amount of about 0.01% to about 0.5%, or even in an amount of about 0.05% to about 0.25% or about 0.045% to about 0.1%. In accordance with another exemplary embodiment, the active ingredient(s) also include zinc gluconate. In one case, zinc gluconate is present in an amount of about 0.001% to about 2%, in an amount of about 0.01% to about 1.5%, or even in an amount of about 0.05% to about 1.25%.

In accordance with an exemplary embodiment, the composition also includes a thickener to obtain the desired viscosity. Suitable thickening agents include substances which increase the viscosity of the composition, cause the composition to gel or coagulate, or the like, such as food-grade or pharmaceutical-grade thickeners, including, for example, hydroxyethylcellulose, hydroxypropyl methylcellulose, carrageenan, guar gum, methylcellulose, methyethylecellulose, acceptable non-ionic thickeners, and the like. The thickener may be present in an amount of about 0.01% to about 10%, in an amount of about 0.1% to about 7%, or even in an amount of about 1% to about 5% or about 0.5% to about 3%.

Composition 102 may also include a humectant such a glycerin, which may be present in an amount of about 0.01% to about 15%, preferably about 0.1% to about 10%, and more preferably about 1% to about 7%. When used, the humectant may facilitate maintaining composition 102 in a liquid form and may help maintain a desired viscosity. In accordance with specific aspects, glycerin facilitates maintaining one or more of the active ingredients in an ionic form and/or facilitates the transport of the active ingredients through composition 102.

The composition may also include a diluent. Exemplary diluents suitable for use with the present composition include Sorbitol, Xylitol, Mannitol, water, alcohols, and oils. In accordance with particular examples of the invention, the composition includes purified water in an amount of about 80% to about 99%, preferably about 85% to about 95%, and more preferably about 88% to about 92%.

The composition may also include sugar alcohols such as sorbitol and xylitol, monnital, lactitol, and the like that act as a sweetener and also as a humectant and/or emulsifier and/or diluent. When used, sorbitol or other sugar alcohol can be present in an amount of about 0.001% to about 0.5%, in an amount of about 0.01% to about 0.1%, or even in an amount of about 0.025% to about 0.075%. Compositions in accordance with the invention may alternatively include a greater percentage of sugar alcohol(s).

The composition may also include a natural or artificial sweetener such as cyclamates, sucralose, saccharin (e.g., sodium or calcium), ace-k, or aspartame which, when included in the composition, can be present in an amount of about 0.001% to about 1.5%, in an amount of about 0.01% to about 1%, or even in an amount of about 0.25% to about 0.75%.

Colorants may also be added to the composition. For example, the composition can include colorants, such that when the composition is applied to or proximate the gingiva, the composition has a color indicative of healthy gingiva—e.g., the composition can be pink in color. Such a composition having a color indicative of healthy gingiva can provide added incentive to users to continue using the composition, which in turn promotes improved health care and hygiene. Colorants may be present in any desired amount. For example, the colorants may include Red #33 and/or Red #40, available from Pylam in an amount of about 0.000005% to about 1%, preferably about 0.00050% to about 0.5%, and more preferably about 0.001% to about 0.1%. Additionally or alternatively, colorants, which are indicative of flavor may be added to the composition. Examples include FD&C Blue #1, FD&C Green #5, FD&C Yellow #5, and FD&C Yellow #6.

Composition 102 may also include other additives or flavorants such as cinnamon oil, clove oil, mints, anise, citrus, fruits, and the like, which, when included in the formula are present in an amount of about 0.01% to about 2%, in an amount of about 0.01% to about 1%, or even in an amount about of about 0.1% to about 0.5%.

Essential oils such as cinnamon bark oil and clove bud oil may be particularly advantageous because they exhibit additional desirable qualities. For example, cinnamon bark oil exhibits antibacterial, antiseptic, antiviral, antispasmodic, antifungal, sedative and analgesic properties and clove bud oil has local anesthetic, antiseptic, antibacterial, and stimulating properties.

In accordance with various embodiments, composition 102 is configured to maintain the composition in contact with a surface for an extended period of time, which has several advantages over traditional compositions. Composition 102 preferably exhibits good microadhesion, and moreover, composition 102 preferably is quite viscous. As such, in general, relatively small amounts of composition 102 and consequently the active agent(s) can be used to effectively provide oral health care or treatment. Additionally, this configuration allows for relatively select placement of the composition on a surface.

Exemplary compositions have a viscosity greater than about 20,000 cp, preferably greater than 30,000 cp, and more preferably greater than about 35,000 cp. By way of more particular examples, the viscosity of compositions range from about 20,000 cp to about 250,000 cp, preferably about 25,000 cp to about 100,000 cp, and more preferably about 30,000 cp to about 50,000 cp, and yet more preferably about 35,000 cp to about 45,000 cp. The viscosity values as set forth herein are measured using a Brookfield, Model DV-II+Pro viscometer, spindle # 6, 10 RPM for 90 seconds at 25 C.

In accordance with other exemplary embodiments, composition 102 includes multiple active ingredients in a carrier. For example, composition 102 can include a plurality of any active ingredients and a carrier in the weight percents disclosed herein. Composition 102 may also include any of the optional ingredients, such as thickeners, sweeteners, additives, flavorants, and colorants as set forth herein. For example, in accordance with an exemplary embodiment, composition 102 includes CPC and zinc gluconate as the active ingredients, wherein the CPC is present in an amount of about 0.001% to about 1%, in an amount of about 0.01% to about 0.5%, or even in an amount of about 0.05% to about 0.25% or about 0.045% to about 0.1%; and wherein the zinc gluconate is present in an amount of about 0.001% to about 1.5%, in an amount of about 0.01% to about 1.0%, or even in an amount of about 0.05% to about 0.75%. In accordance with additional embodiments, the composition includes zinc and/or CPC, for example in the quantities noted above, and one or more additives as a second active ingredient.

In accordance with other exemplary embodiments, composition 102 includes one or more active ingredients and a colorant indicative of healthy gingiva, wherein the color of composition 102 is more than merely decorative; it also serves the function of encouraging those that use the product to continue to use the product because there is an immediate appearance, upon application of composition 102, that healthy gingival is achieved.

A pH of composition 102 may vary in accordance with a particular application. In accordance with various embodiments of the invention, the pH is between about 4-10, preferably about 4-7, and more preferably about 5-5.4.

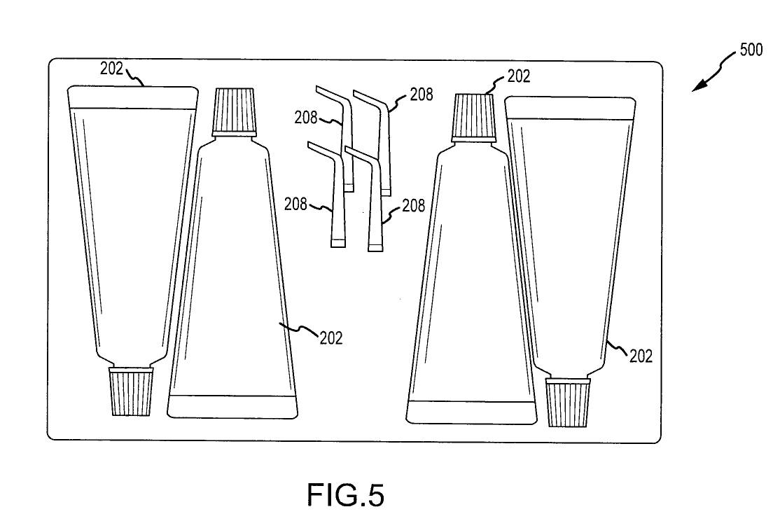

FIG. 5 illustrates a kit 500, including multiple systems, in accordance with yet additional embodiments of the invention. As illustrated, kit 500 includes four systems; however, kits in accordance with other embodiments of the invention may include a different number, e.g., 1, 2, 4, 7, 10, or the like number of systems.

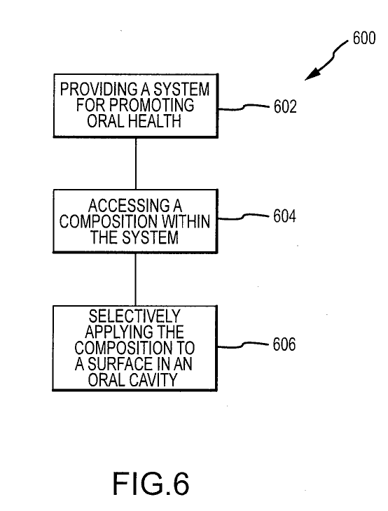

In accordance with further exemplary embodiments, with reference to FIG. 6, as well as continuing reference to 1-5, a method 600 of facilitating oral health care includes providing a system (e.g., system 100), including a composition (e.g., composition 102) (step 602) accessing the composition within the container (step 604), and selectively applying the composition to an oral cavity surface (step 606).

Providing a system in accordance with step 602 can include any method now known or hereinafter devised for filling a container with a fluid. With reference to FIG. 1, composition 102 can suitably be filled in one end of container 104 and then sealed to maintain composition 102 within container 104. Alternatively, with reference to FIG. 2, vessel 202 may be filled with composition 102 and then cap 206 sealably attached to vessel 202. In addition, providing composition 102 within container 104 may include providing a single and/or daily dose of composition 102, or multiple doses of composition 102.

Accessing composition 102 within container 104 suitably comprises removal of an access or closure device or component from container 104. For example, in accordance with an exemplary embodiment, accessing composition 102 within container 104 comprises detachably removing an access component, e.g., end portion 122, to provide an access to the composition.

Selectively applying composition 102 to a surface can suitably include expulsing or otherwise forcing or delivering composition 102 from a vial 110 to applicator portion 108 of container 104. For example, in accordance with an exemplary embodiment, composition 102 can be “squeezed” from an expulsion portion 106, into an applicator portion 108, through an applicator tip 118 and onto a targeted region of oral cavity surface. In accordance with various embodiments of the invention, the system does not require any additional additives or solutions for use. That is, the compositions can be applied directly to a surface without diluting or the like. Further, the compositions can be applied without additional devices such as cups or the like.

Regular and/or systematic use of systems in accordance with various embodiments of the invention onto one or more surfaces results in improved oral health.

A method of forming a composition in accordance with various embodiments of the invention includes the steps of adding a humectant (e.g., glycerin) to a first mixing vessel and the adding a thickener (e.g., hydroxyethylcellulose) to the humectant and mixing until a uniform, lump-free slurry forms. The slurry should not sit for too long at this stage, or it may become cement-like in texture and viscosity. In a second mixing vessel, add a diluent (e.g., water) and add the humectant/thickener slurry slowly (over a period of a few hours) to the diluent and mix until a smooth mixture is obtained. Once the gum is hydrated, add any sugar alcohol, sweetener, and colorant to the mixture and mix until each is dissolved. Then, add a preservative and mix until dissolved and uniform. Finally, add the actives and any oils and mix until the actives and oils are dispersed in the solution.

### SPECIFIC EXAMPLES

The following non-limiting examples illustrate improvement in oral health using a system, kit, and method in accordance with various embodiments of the invention. These examples are merely illustrative, and it is not intended that the invention be limited to the examples. Systems in accordance with the present invention may include the ingredients listed below as well as additional and/or alternative inert materials, preservatives, and other constituents typically found in compositions for promoting oral health. In the case where exemplary inert materials and/or preservatives are listed, these ingredients are merely exemplary, and it is understood that other similar ingredients may be substituted for the materials listed in the examples below.

### Example 1

A pale light pink viscous gel, having a viscosity of about 40,000 cp, with cinnamon-clove characteristic odor and taste is formed by admixing the following ingredients, as described above, in the amounts shown. The composition was sealed in system 400, illustrated in FIG. 4.

TABLE 1

| | | | Exemplary |
|---|---|---|---|
| Ingredient | Supplier | Weight % | Wt % Range |
| Purified Water | Copacker | 91.504 | 80-99 |
| Glycerin USP | Acme-Hardesty | 5.000 | 0.01-15 |
| HEC 250 HX | Hercules-Aqualon | 2.000 | 0.01-10 |
| Sorbitol | Roquette | 0.050 | 0.001-0.5 |
| Sucralose | Tate & Lyle | 0.400 | 0.001-1.5 |
| Cetylpyridinium | Dishman | 0.100 | 0.001-1 |
| Chloride | Pharmaceuticals | | |
| Zinc Gluconate | American | 0.592 | 0.001-1.5 |
| USP | International | | |
| Cinnamon Bark | Spectrum | 0.250 | 0.001-2 |
| (Oil) | | | |
| Clove Bud (Oil) | Spectrum | 0.005 | 0.001-2 |
| Red #40 (1% sol.) | Pylam | 0.099 | 0.000005-1 |

A clinical study, including 24 subjects, evaluating the efficacy of the composition of Table 1 was conducted. Subjects were scored at baseline, three weeks, and at six weeks using the Low and Silness Gingival Index (1963) (GI) and the Turesky Modification (1970) of the Quigley Hein Plaque Index (1962) (PI) indices. Plaque and calculus quality, thickness, maturity, and mass were also observed at these times.

At the end of a three-week period, there was an observed lessening or reduction of plaque quality, thickness, mass, and maturity; the lessening was greater (greater decrease) for those using the composition of Table 1, compared to a placebo. The observance that plaque quality was reduced is important because the presence of actively growing plaque bacteria (biofilm) is important in the development of inflammation, which leads to gingivitis and periodontitis.

In addition, at the end of the three-week period a lessening of the quality of calculus was also observed. A general improvement of gingival health was also observed. An extremely thin, slightly detached layer of epithelial cells was also found on the surface of attached gingival surfaces at the marginal ridge, close to the areas where plaque and tartar was likely disrupted off the teeth with subjects using the composition of Table 1, which indicated promotion of faster healing of gingival tissues.

Subjects at the end of the six-week period showed a statistically significant (p-value=0.05) greater performance than a placebo in both absolute (0.741) and percentage (40%) improvement in GI. There was also a directional improvement for PI in the absolute (1.805) and percent (49%) improvement for those using the formula of Table 1 compared to a placebo. There was also approximately a 50% reduction in plaque thickness at the end of the six-week period in about 73% of the subjects that were checked for plaque quality or thickness.

### Example 2

A pale light pink viscous gel, having a viscosity of about 40,000 cp, with cinnamon-clove characteristic odor and taste is formed by admixing the following ingredients, as described above, in the amounts shown. The composition was sealed in system 400, illustrated in FIG. 4.

TABLE 2

| | | | Exemplary |
|---|---|---|---|
| Ingredient | Supplier | Weight % | Wt % Range |
| Purified Water | Copacker | 92.096 | 80-99 |
| Glycerin USP | Acme-Hardesty | 5.000 | 0.01-15 |
| HEC 250 HX | Hercules-Aqualon | 2.000 | 0.01-10 |
| Sorbitol | Roquette | 0.050 | 0.001-0.5 |
| Sucralose | Tate & Lyle | 0.400 | 0.001-1.5 |
| Cetylpyridinium | Dishman | 0.100 | 0.001-1 |
| Chloride | Pharmaceuticals | | |
| Cinnamon Bark | Spectrum | 0.250 | 0.001-2 |
| (Oil) | | | |
| Clove Bud (Oil) | Spectrum | 0.005 | 0.001-2 |
| Red #40 (1% sol.) | Pylam | 0.099 | 0.000005-1 |

A clinical study, including 22 subjects, evaluating the efficacy of the composition of Example 2 was conducted. The Low and Silness Gingival Index (1963) (GI) for subjects using the formula of Table 2 for a six-week period showed a greater performance than a placebo in both absolute (0.405) and percentage (22%) improvement in GI. There was also a directional improvement for the Turesky Modification (1970) of the Quigley Hein Plaque Index (1962) (PI) in the absolute (1.489) and percent (41%) improvement for those using the formula of Table 2 compared to a placebo.

### Example 3

A clinical study comparing V-MI scores of subjects using the composition of Table 1 was conducted to evaluate calculus dissolution. During the three-month study, the subjects were instructed to brush with toothpaste twice daily and apply the composition prior to retiring. The subjects did not receive a professional cleaning just prior to the study, which evaluated the efficacy of the composition to remove tartar.

For 18 subjects using a system including the composition of Table 1, there was a statistically significant difference between original V-MI scores and V-MI scores (51%) after three months of treatment with the product, which indicates the system is effective at calculus dissolution.

### Example 4

In another clinical study, 50 subjects received professional prophylaxis, including scaling and polishing, to remove supragingival calculus, extrinsic stain, and plaque deposits from the mandibular anterior teeth and initial V-MI measurements at 18 VM sites per subject were recorded prior to the study to evaluate calculus inhibition. Subjects were requested to refrain from flossing the mandibular 6 anterior teeth and place the composition between and around those teeth using system 400, illustrated in FIG. 4. At the end of a three-month period, VM-I measurements for the same teeth were recorded and analyzed using analysis of covariance. The V-MI scores at the end of the three-month period were statistically significantly lower (59%) than the initial V-MI scores. The study indicated that the system was effective at Calculus inhibition.

### Example 5

A clinical study comparing V-MI scores of 40 subjects using the composition of Table 1 was conducted to evaluate disruption or dissolution of existing calculus bridges. During a three-month study, the subjects were instructed to brush with toothpaste twice daily and apply the composition prior to retiring.

At the end of the three-month study, a statistically significant difference (30% difference) of before and after scores of the subjects using the composition of Table 1 was observed. In addition, a statistically significant difference (26% difference) of the 40 subjects compared to 40 subjects using a placebo was observed, and a statistically significant difference (99%) between the change in scores before and after the study between the placebo group and the group using a system including the composition of Table 1.

### Example 6

A clinical study comparing Gingivitis Index (GI), Plaque Index (PI), and Bleeding Index (BI) scores using the composition of Table 1 was conducted. Forty-five subjects were evaluated over a period of three months and 39 subjects were evaluated over a period of six months. During the study, the subjects were instructed to brush with toothpaste twice daily and apply the composition prior to retiring. The subjects were asked not to use other oral hygiene products. After a baseline examination, all subjects received dental prophylaxis.

At the end of the three-month period, a 16% reduction in GI was observed, a 26% reduction in PI was observed, and a 68% reduction in BI was observed. At the end of the six-month period, a 32% reduction in GI was observed, a 33% reduction in PI was observed, and a 78% reduction in BI was observed. All reductions are statistically significant (p-Value<0.01) using the Mann-Whitney Rank Sum Test.

### Example 7

A pale light pink viscous gel, having a viscosity of about 40,000 cp, with cinnamon-clove characteristic odor and taste is formed by admixing the following ingredients, as described above, in the amounts shown. The composition was sealed in system 400, illustrated in FIG. 4.

TABLE 3

| | | | Exemplary |
|---|---|---|---|
| Ingredient | Supplier | Weight % | Wt % Range |
| Purified Water | Copacker | 91.604 | 80-99 |
| Glycerin USP | Acme-Hardesty | 5.000 | 0.01-15 |
| HEC 250 HX | Hercules-Aqualon | 2.000 | 0.01-10 |
| Sorbitol | Roquette | 0.050 | 0.001-0.5 |
| Sucralose | Tate & Lyle | 0.400 | 0.001-1.5 |
| Zinc Gluconate | American | 0.592 | 0.001-1.5 |
| USP | International | | |
| Cinnamon Bark | Spectrum | 0.250 | 0.001-2 |
| (Oil) | | | |
| Clove Bud (Oil) | Spectrum | 0.005 | 0.001-2 |
| Red #40 (1% sol.) | Pylam | 0.099 | 0.000005-1 |

A clinical study comparing Gingivitis Index (GI), Plaque Index (PI), and Bleeding Index (BI) scores using the composition of Table 3 was conducted. Fifty-one subjects were evaluated over a period of three months and forty-three people were evaluated over a period of six months. During the study, the subjects were instructed to brush with toothpaste twice daily and apply the composition prior to retiring. The subjects were asked not to use other oral hygiene products. After a baseline examination, all subjects received dental prophylaxis.

At the end of the three-month period, a 11% reduction in GI was observed, a 17% reduction in PI was observed, and a 58% reduction in BI was observed. At the end of the six-month period a 16% reduction in GI was observed, a 21% reduction in PI was observed, and a 52% reduction in BI was observed. All reductions are statistically significant (p-Value<0.01) using the Mann-Whitney Rank Sum Test.

### Example 8

A clinical study comparing Gingivitis Index (GI), Plaque Index (PI), and Bleeding Index (BI) scores using the composition of Table 2 was conducted. Fifty subjects were evaluated over a period of three months and 44 people were evaluated over a period of six months. During the study, the subjects were instructed to brush with toothpaste twice daily and apply the composition prior to retiring. The subjects were asked not to use other oral hygiene products. After a baseline examination, all subjects received dental prophylaxis.

At the end of the three-month period, a 18% reduction in GI was observed, a 25% reduction in PI was observed, and a 65% reduction in BI was observed. At the end of the six-month period, a 31% reduction in GI was observed, a 32% reduction in PI was observed, and a 75% reduction in BI was observed. All reductions are statistically significant (p-Value<0.01) using the Mann-Whitney Rank Sum Test.

### Example 9

A clinical study comparing V-MI scores using a placebo and the compositions of Tables 1 and 4 was conducted. Data was recorded at baseline, 40 days and 90 days. Subjects were provided coded tubes of the gel formulation to which they were assigned. The subjects were instructed to apply the gel daily, once before going to bed. The gel was applied between the teeth at the gum margin all along the arch. After applying, the subjects were instructed to spit out any excess and not to eat or drink prior to an extended period of rest.

TABLE 4

| | | | Exemplary |
|---|---|---|---|
| Ingredient | Supplier | Weight % | Wt % Range |
| Purified Water | Copacker | 91.504 | 80-99 |
| Glycerin USP | Acme-Hardesty | 5.000 | 0.01-15 |
| HEC 250 HX | Hercules-Aqualon | 2.000 | 0.01-10 |
| Sorbitol | Roquette | 0.050 | 0.001-0.5 |
| Sucralose | Tate & Lyle | 0.400 | 0.001-1.5 |
| Cetylpyridinium | Dishman | 0.100 | 0.001-1 |
| Chloride | Pharmaceuticals | | |
| Zinc Gluconate | American | 1.184 | 0.001-1.5 |
| USP | International | | |
| Cinnamon Bark | Spectrum | 0.250 | 0.001-2 |
| (Oil) | | | |
| Clove Bud (Oil) | Spectrum | 0.005 | 0.001-2 |
| Red #40 (1% sol.) | Pylam | 0.099 | 0.000005-1 |

After 90 days, users of the composition of Table 4 had 19% better V-MI scores, and users of the composition of Table 1 had 10% better V-MI scores. Following treatment, users of the composition of Table 1 had 0.31 better absolute V-MI scores and users of the composition of Table 4 had 0.32 better absolute V-MI scores.

The present invention has been described above with reference to various exemplary embodiments. However, those skilled in the art will recognize that changes and modifications may be made to the exemplary embodiments without departing from the scope of the present invention. For example, the various operational steps, as well as the components for carrying out the operational steps, may be implemented in alternate ways depending upon the particular application or in consideration of any number of cost functions associated with the operation of the system, e.g., various of the steps may be deleted, modified, or combined with other steps. These and other changes or modifications are intended to be included within the scope of the present invention, as set forth in the following claims.

We claim:

1. A system to improve oral health, the system comprising:
a gel composition including an active ingredient for improving oral health, the gel composition having a viscosity greater than about 20,000 cp; and
a container comprising a resilient portion and an applicator portion, wherein the container is configured to directly apply the composition to a surface within an oral cavity.

2. The system to improve oral health according to claim 1, wherein the container comprises a single dose of composition and is configured for discarding after application of said single dose of composition.

3. The system to improve oral health according to claim 1, wherein the container comprises multiple doses of composition and is configured for discarding after application of the multiple dose of composition.

4. The system to improve oral health according to claim 1, wherein the container comprises a vessel portion and a spout portion.

5. The system to improve oral health according to claim 4, wherein the vessel portion and the spout portion are threadedly engaged.

6. The system to improve oral health according to claim 4, wherein the vessel portion and the spout portion are rotatably attached.

7. The system to improve oral health according to claim 1, wherein the composition comprises zinc salt.

8. The system to improve oral health according to claim 1, wherein the composition comprises zinc gluconate and cetylpyridinium chloride.

9. The system to improve oral health according to claim 1, wherein the composition comprises cetylpyridinium chloride.

10. The system of claim 1, wherein the viscosity of the composition is about 25,000 cp to about 100,000 cp.

11. The system of claim 1, wherein the viscosity of the composition is about 30,000 cp to about 50,000 cp.

12. A disposable, self-contained oral health care system configured to reduce at least one of tartar, plaque, gingivitis and gum diseases within an oral cavity, the system comprising:
a composition configured to reduce at least one of tartar, plaque, gingivitis and gum diseases within the oral cavity, said composition being configured as a single-dosage; and
a container configured for sealed storage of the composition, said container having a vessel portion and a spout portion, the container further configured for forcible expulsion and selective application of the composition to an oral cavity surface.

13. The system of claim 12 wherein the composition has a viscosity of greater than about 20,000 cp.

14. The system of claim 12, wherein said composition comprises an active ingredient selected from the group consisting of cetylpyridinium chloride, dicalcium phosphate dehydrate, hydrogen peroxide, sanguinaria extract, sodium bicarbonate, sodium lauryl sulfate, stannous fluoride, zinc salts alkyl dimethyl amine oxide, alkyl dimethyl glycine, eucalyptol, menthol, methyl salicylate, thymol, sodium citrate, peppermint oil, sage oil, polymethylsiloxane, polxamer, and stannous pyrophosphate.

15. The system of claim 14, wherein the active ingredient comprises cetylpyridinium chloride.

16. The system of claim 14, wherein the active ingredient comprises zinc salt.

17. The system of claim 14, wherein the active ingredient comprises zinc gluconate.

18. An oral health care system configured to improve oral health, the system comprising:
a composition comprising multiple active ingredients to improve oral health; and
a container configured for sealed storage of the composition, said container having a vessel portion and a spout portion, the container further configured for forcible expulsion and selective application of the composition to an oral cavity surface.

19. The oral health care system of claim 18, wherein the composition has a viscosity greater than about 20,000 centipoise.

20. The system of claim 19, wherein the viscosity of the composition is about 25,000 cp to about 100,000 cp.
